(19) United States
(12) Patent Application Publication
(10) Pub. No.: US 2023/0001223 A1
(43) Pub. Date: Jan. 5, 2023

(54) CLINICAL AND PERSONAL ELECTROMAGNETIC STIMULATOR AND IONTOPHORESIS DEVICE FOR TREATING RETINA AND OPTICAL NERVE DISEASES

(71) Applicant: BIORETINA BIYOTEKNOLOJI VE BIYOMEDIKAL SAGLIK YATIRIMLARI ANONIM SIRKETI, Ankara (TR)

(72) Inventors: Umut ARSLAN, Ankara (TR); Emin OZMERT, Ankara (TR)

(73) Assignee: BIORETINA BIYOTEKNOLOJI VE BIYOMEDIKAL SAGLIK YATIRIMLARI ANONIM SIRKETI, Ankara (TR)

(21) Appl. No.: 17/777,643

(22) PCT Filed: Dec. 6, 2019
(86) PCT No.: PCT/TR2019/000086

§ 371 Date: May 18, 2022

(30) Foreign Application Priority Data
Nov. 19, 2019 (TR) 2019/18027

(51) Int. Cl. A61N 2/00; A61N 2/02; A61N 1/32

(52) U.S. Cl. CPC A61N 2/006; A61N 2/02; A61N 2/002; A61N 1/325

(57) ABSTRACT

An electromagnetic stimulator and iontophoresis device used in treating retina and optical nerve diseases is provided. The electromagnetic stimulator and iontophoresis device enables to reach required threshold values for treating the retina and optical nerve diseases with magnetic fields to eliminate a thermal damage risk formed at a cellular level by cooling cages closed over electromagnetic coils, and allows usage by a patient alone at home, who has low vision, without receiving any professional help by an audio command guidance system including a speaker.

### CROSS REFERENCE TO THE RELATED APPLICATIONS

This application is the national stage entry of International Application No. PCT/TR2019/000086, filed on Dec. 6, 2019, which is based upon and claims priority to Turkish Patent Application No. 2019/18027 filed on Nov. 19, 2019, the entire contents of which are incorporated herein by reference.

### TECHNICAL FIELD

The invention is related to electromagnetic stimulator and iontophoresis devices used for treating retina and optical nerve diseases.

The invention is particularly related to a personal electromagnetic stimulator and iontophoresis device which enables to reach the required threshold values for treating retina and optical nerve diseases with very low magnetic fields, which eliminates thermal damage risk formed at the cellular level by means of cooling cages, enables the transfer of ocular drug injections made to the retina using the electromagnetic iontophoresis method and allows usage by the patient alone at home, who has low vision, without receiving any professional help, thanks to the audio command guidance system.

### BACKGROUND

Chronic retina and optical nerve diseases can be ischemic or degenerative. In degenerative diseases, the synthesis of the growth factors which enable vital functions of retina nerve cells are damaged genetically. In ischemic patients however, the blood flow and supply to retina nerve cells are damaged. In both cases, chronic and progressive visual loss is experienced and in the end legal blindness occurs.

The stimulation of the retina and optical nerve electromagnetically leads to retina blood flow increase at a cellular level, increase in vascular permeability and increase of receptor sensitivity in growth factors. Simultaneous stimulation of the occipital cortex leads to an increase in neurotrophic growth factor levels in the retina. The simultaneous electromagnetic stimulation of the retina, optical nerve, optical tracts and visual cortex leads to cellular depolarization and phosphene formation. In order for all of these effects to be realized a suprathreshold electromagnetic field is required. The required suprathreshold magnetic field can be obtained by the correct positioning of electromagnetic coils, correct frequency, power, intensity and time. These effects that are reparative in retina and optical nerve diseases must be applied at certain periods and times mostly for a lifelong period in chronic diseases. Due to this reason, in order to obtain an efficient result, the patient needs to use an electromagnetic stimulator on his/her own at home continuously and correctly.

In order for electromagnetic stimulation to be effective in retina and optical nerve diseases, the occipital cortex, inter-hemispherical sulcus, and the retina and optical nerves need to be stimulated at the same time. Retina and optical nerve stimulation is carried out from a vertex distance which is the most efficient distance. The vertex distance, is the closest distance to the cornea apex which does not prevent the closing of the eyelids. However the electromagnetic stimulator devices in the prior art, may cause thermal damage at a cellular level and therefore it is not possible to stimulate the eyeball, retina and optical nerve from the vertex distance. At the same time the devices of the prior art are suitable to be used under the supervision of a health personnel. The incorrect usage applied under the supervision of people who are not experts, may lead to many complications including visual loss. Some drug injections are applied in treating retina diseases. In order for these drugs to reach the retina, an iontophoresis procedure is required. In order for an iontophoresis procedure to be electromagnetically efficient the coils need to be effective simultaneously, in the same way, in the correct position and at the correct distance. In order to establish correct stimulation points and correct electrical polarization, the device needs to have a standard design. The present iontophoresis devices have effect not with an electromagnetic field but with an electric current. Electromagnetic iontophoresis is designed such that it does not contact the eye and therefore it is much safer than electrical iontophoresis devices.

The document numbered US2017165485 can be given as an example to the stimulator device in literature. In said document a device that has been developed to treat visual complaints during headaches and migraines is described. It is not possible to stimulate the eyeball, retina and optical nerves from the vertex distance with said device, it is possible to stimulate the supraorbital region at most with this device. It is not possible to reach effective suprathreshold values in the eye, during the treatment of retina and optical nerves with this device that is not indicated for treating degenerative and ischemic retina optical nerve diseases. The device does not have any cooling mechanism in order for the device to be used from the vertex distance. The device does not have a standard cap system placement design to the correct poles in order to perform electromagnetic iontophoresis. This design that has been defined with general terms according to requirements for placement of electromagnetic coils and electrodes does not have the required standard design in order to perform iontophoresis. This device does not comprise a design having the standard shape, number, distance and parameters that can apply an electromagnetic field at the vertex distance, and to establish standard poles between other electromagnetic coils.

Another example is the document numbered WO2009063435. Said document is related to a sensor system which shows if the coils have been placed in the correct position on the occipital cortex during amblyopia treatment. It is not indicated for being used in treating retina and optical nerve diseases. The device does not have any cooling mechanism in order for the device to be used from the vertex distance. The device does not have a standard cap system placement design to the correct poles in order to perform electromagnetic iontophoresis. Therefore said system is not designed to stimulate the eyeball, retina and optical nerves from the vertex distance.

Another example is the document numbered US2009099623. Said document is related to the treatment of medical conditions associated with the central nervous system and to increasing cognitive functions. The device does not have any cooling mechanism in order for the device to be used from the vertex distance. The device does not have a standard cap system placement design to the correct poles in order to perform electromagnetic iontophoresis. Said device is not designed or indicated for treating degenerative and ischemic retina optical nerve diseases.

Another example is the document numbered US2013137918. Said document is related to a low frequency magnetic stimulation treatment device. The device does not have any cooling mechanism in order for the device to be used from the vertex distance. The device does not have a standard cap system placement design to the correct poles in order to perform electromagnetic iontophoresis. Said device is not designed or indicated for treating degenerative and ischemic retina optical nerve diseases.

Another example is the document numbered GB2295093. Said document is related to a synchronized electromagnetic therapy device. The device does not have any cooling mechanism in order for the device to be used from the vertex distance. The device does not have a standard cap system placement design to the correct poles in order to perform electromagnetic iontophoresis. Said device is not designed or indicated for treating degenerative and ischemic retina optical nerve diseases.

As a result, the presence of the above mentioned problems and the inadequacy of the present solutions has made it a necessity to provide a development in the related technical field.

### SUMMARY

The present invention is related to a personal electromagnetic stimulator and iontophoresis device for treating retina and optical nerve diseases which eliminate the above mentioned disadvantages and bring about new advantages.

The aim of the invention is reach the required threshold values with very low magnetic fields in order to treat retina and optical nerve diseases by means of the cap worn by the patient, comprising electromagnetic coils that has a design which can be correctly positioned and which can stimulate the eyeball, retina and optical nerves directly.

The aim of the invention is to eliminate the risk of thermal damage at the cellular level as the electromagnetic coils are covered with cooling cages.

Another aim of the invention is to increase the passage of the ocular drug injections to the retina by means of the electromagnetic iontophoresis method as the electromagnetic coils are connected to the cap system such that it allows correct polarization.

Another aim of the invention is to prevent the formation of opacities in the cornea, lens and vitreous and to prevent permanent visual loss by eliminating thermal damage risk at the cellular level.

Another aim of the invention is to enable correct, safe and efficient usage of the stimulator device by the patient alone, without requiring professional assistance by means of the audio commands provided in different languages, to the patient having visual impairment.

Another aim of the invention is to notify the doctor following up the patient if the patient is using the device correctly or not, by means of the counter system on the device.

The invention is related to a stimulator and iontophoresis device used for treating retina and optical nerve diseases, in order to carry out all of the aims that can be construed from the information provided above and the detailed description below.

The invention is characterized in that it comprises;
a cap made of two parts; one being the front cap (10a) comprising an electromagnetic coil assembly including electromagnetic coils which create 4 electromagnetic fields at a vertex distance to the eyeballs, that have been covered with a cooling cage, and the other being the rear cap comprising an electromagnetic coil assembly including electromagnetic coils which create 4 electromagnetic fields that stimulate the occipital cortex region and 1 electromagnetic field that stimulates the inter-hemispheric region, that have been covered with a cooling cage,
wherein said device comprises a cooling compressor connected with cooling pipes that pass through the connection pipe that is connected with the cooling cages inside the front cap and rear cap and gas perfluorocarbon tank, an electronic card and a processor having a counter thereon which adjusts the intensity, frequency and time of the electromagnetic field created by the electromagnetic coils, an electronic control box having a speaker connected to the electronic card and processor.

The structural and characteristic features of the invention and all of its advantages can be better understood by means of the detailed description and by referring to the attached figures. Due to this reason, if an evaluation needs to be carried out, it must be done so by taking into consideration the attached figures and the detailed description.

### BRIEF DESCRIPTION OF THE DRAWINGS

FIG. 1 General view of the electromagnetic iontophoresis device subject to the invention.

FIG. 2 The perspective view of the internal detail of the stimulator and iontophoresis device cap subject to the invention.

FIG. 3 The perspective view of the stimulator and iontophoresis device cap subject to the invention.

FIG. 4 Detailed view of the electromagnetic coil assembly of the stimulator and iontophoresis device subject to the invention.

FIG. 5 The detailed below of the stimulator and iontophoresis device cap and the electronic control box subject to the invention.

### DESCRIPTION OF THE PART REFERENCE NUMBERS

10. Cap
10a. Front Cap
10b. Rear Cap
11. Connection rail
12. Electromagnetic coil assembly
121. Electromagnetic coil
1211. LED indicator
1212. Magnetic field sensor
122. Cooling cage
1221. Cooling tank
1222. Gas inlet hole
1223. Gas outlet hole
123. Electric cable
124. Cooling pipe
13. Sponge rubber
20. Electronic control box
21. Electronic card and processor
211. Counter
22. Cooling compressor
23. Gas perfluorocarbon tank
24. Screen
25. Speaker
26. Control panel
27. Power Supply
28. On/off button
30. Connection pipe
A. Stimulator and iontophoresis device

### DETAILED DESCRIPTION OF THE EMBODIMENTS

In this detailed description the preferred embodiments of the stimulator and iontophoresis device (A) subject to the invention has been described only in order to better explain the subject matter and such that it does not create any kind of limiting effect.

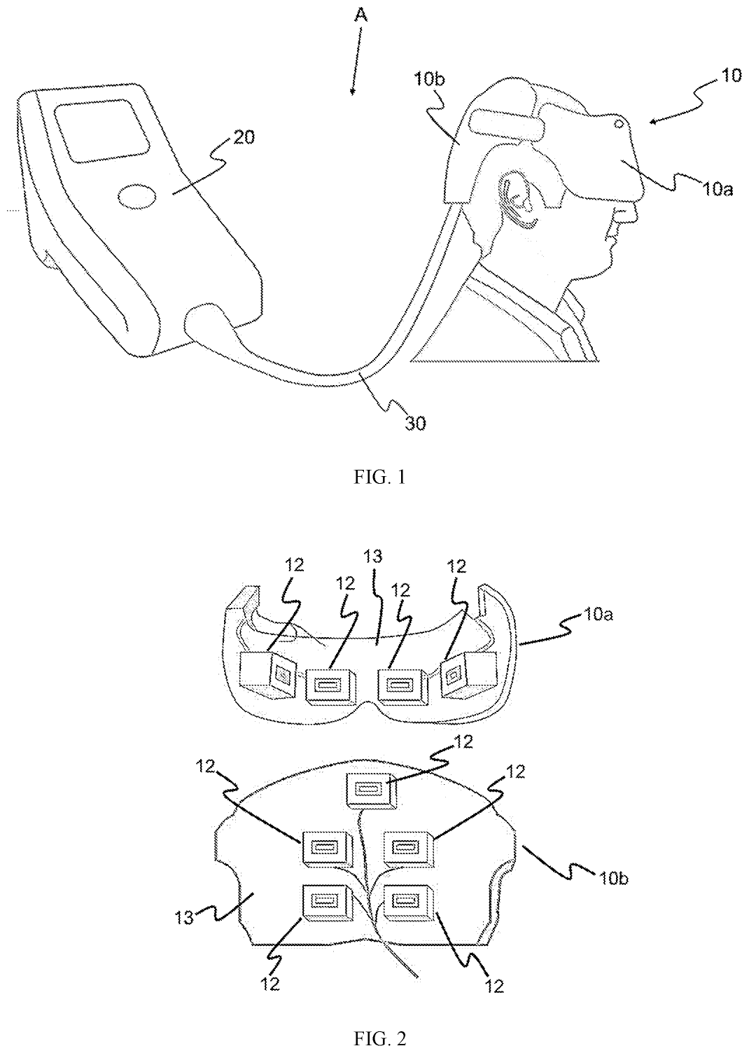

The general view of the stimulator and iontophoresis device (A) subject to the invention has been provided in FIG. 1. The stimulator and iontophoresis device (A) according to the invention basically comprises; a cap (10) made of two parts, one being the front cap (10a) and the other being the rear cap (10b) connected to each other by a connection rail (11) having an electromagnetic coil assembly (12) which enables to reach the required threshold values in order to treat retina and optical nerve diseases with very low magnetic fields, and an electronic control box (20) connected via the connection pipe (30) to the cap (10) having electric cables (123) and cooling pipes (124) passing therein.

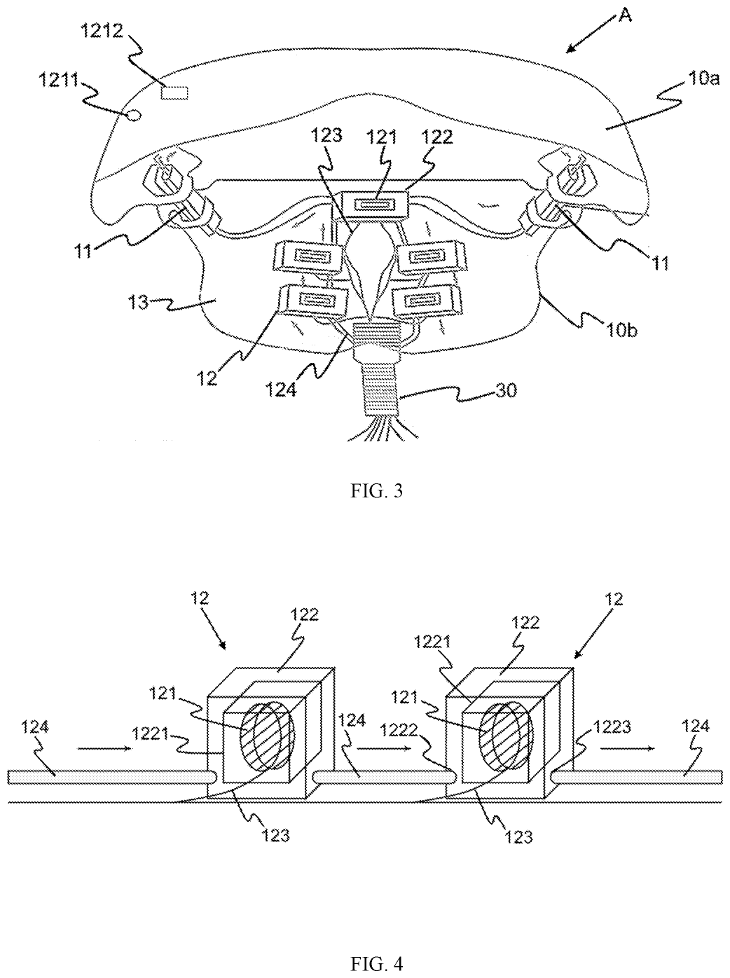

As it can be seen from FIGS. 2 and 3, the cap (10) that is worn over the head of the patient is made of two parts being the front cap (10a) and the rear cap (10b) connected to each other with a connection rail (11) from their edges. Said front cap (10a) and the rear cap (10b) can be adjusted according to the diameter of the user by being brought closer or by being distanced apart via the connection rail (11). Therefore by means of the adjustable configuration of the cap (10) suitable positioning of the electromagnetic coil assembly (12) has been provided for all head diameters. The front cap (10a) and the rear cap (10b) is made of non-inflammable, antibacterial, antistatic medical plastic, carbon fibre or any other kind of medical material.

The cap (10) comprises a total of 9 electromagnetic coil assemblies (12) that are fixedly embedded in order to reach the required threshold values with very low magnetic fields to treat retina and optical nerve diseases.

The front cap (10a) comprises 4 electromagnetic coil assemblies (12) that create electromagnetic fields at the vertex distance to the eyeballs and the rear cap (10b) comprises a total of 5 electromagnetic coil assemblies (12) that are in contact with the scalp wherein 4 electromagnetic fields stimulate the occipital cortex region and 1 electromagnetic field stimulates the inter-hemispheric region.

The sections besides the electromagnetic coil assembly (12) at the inner surfaces of the front cap (10a) and the rear cap (10b) are covered with a smart medical sponge rubber (13) that is non-inflammable, biocompatible, antiallergic and antibacterial. Said smart sponge rubber (13) enables the electromagnetic coil assemblies (12) to remain in the correct position during usage.

The electromagnetic coil assembly (12) illustrated in FIG. 4, is generally formed of an electromagnetic coil (121) and a cooling cage (122). Said electromagnetic coils (121) have polygonal form, which enables at least 2 electromagnetic coils (121), which stimulate an eyeball, to contact each other with a large surface area and which enables the two electromagnetic coils (121) that are in contact with each other to form a magnetic effective field that reaches the retina and optical nerve depth continuously from a vertex distance. The vertex distance is the distance which does not prevent the closing of the eyelids and which is the distance closest to the cornea apex. Therefore the electromagnetic coils (121) can affect the eyeball completely at the magnetic field depth without touching the eye. Therefore the placement of the electromagnetic coil assemblies (12) in the cap (10), the distance of the coils to each other, the polygonal structure and the intensity of the magnetic field allow the formation of suitable poles for iontophoresis. The arrangement of the electrical connections of the electromagnetic coils (121) are arranged in the front cap (10a) and the rear cap (10b) in order to form a standard and correct polarization on the cap (10); such that random placement is not allowed.

LED indicators (1211) and magnetic field sensors (1222) are provided on each of the electromagnetic coils (121). Therefore the tracking of active and passive electromagnetic coils (121) have been enabled. The magnetic field sensors (1222) enable the transmission of the magnetic field formed, when the electromagnetic coil (121) is active onto the screen (24) of the electronic control box (20). As a result when the stimulator device (A) is active, by which electromagnetic coil (121) the amount of magnetic field is created and if this field is correct or not can be tracked during the duration of the treatment process.

The electromagnetic coils (121) receive their power from the power supply (27) in the electronic control box (20) to which it is connected via electrical cables (123). Said power supply (27) can be an adapter and/or battery-accumulator that operates directly with the electric network. If the stimulator and iontophoresis device (A) is operated with electricity, the battery-accumulator steps in order not to delay the treatment time and the battery-accumulator percentage can be seen on the screen (27).

In order to prevent thermal damage risk at the cellular level that may be encountered due to electromagnetic coils (121), a cooling cage (122) has been covered over each electromagnetic coil (121). The cooling cages (122) are made of fibreglass, fibre carbon or any kind of other medical material, formed as two layered hollow impermeable boxes. Each cooling cage (122) has a cooling tank (1221) which allows gas circulation therein, and a gas outlet hole (1222) and a gas inlet hole (1223) allowing the entrance and discharging of the cooling gas. The cooling compressor (22) in the electronic control box (20) and the elastic cooling pipes (124) extending from the gas perfluorocarbon tank (23) pass through each cooling cage (122) and allow the gas to be pumped into the cooling tank (1221) via the gas inlet hole (1222). The cooling gas exiting out of the cooling tank (1221) via the gas outlet hole (1223) cools the next cooling tank (1221) and then returns to the cooling compressor (22) and the cooling tank (1221) by recirculation.

The electric cables (123) connected to the electromagnetic coils (121) have been isolated below the cooling cage (122) and the cooling pipes (124) used in the cooling process is isolated under sponge rubber (13). Although said electric cables (123) and cooling pipes (124) pass through the connection rails (11), they are formed as an accordion such that they can be extended or shortened. This ensures adjustment ease for each head diameter.

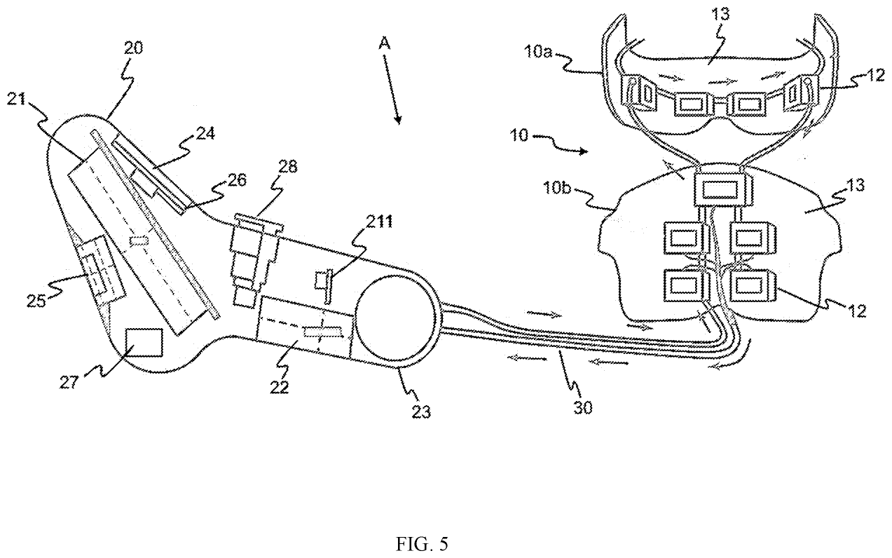

As it can be seen in FIG. 5, the electronic control box (20) connected to the cap (10) via the electric cables (123) passing through the connection pipe (30), includes an electronic card and a processor (21) which adjusts the electromagnetic field intensity, frequency and time created by the electromagnetic coils (121). These parameters cannot be changed by the patient in order for the stimulator device (A) to be used safely and efficiently at home, alone by the patient. The patient can only control the on/off button (28). When the on/off button (28) is pressed, as standard, the stimulator device (A) creates 2000 milligauss electromagnetic field for 30 minutes at 42 hertz frequency from each electromagnetic coil (121). These parameters are the average suprathreshold values that are obtained following clinical trials. The determined frequency and electromagnetic field intensity in order to carry out effective electromagnetic iontophoresis, has been fixed and it cannot be changed by the patient.

In the case that the doctor following up the patient desires to conduct changes in the parameters with an aim besides performing electromagnetic iontophoresis, the technical staff can adjust the magnetic field intensity, frequency and time and which electromagnetic coil (121) needs to be passive-active. This adjustment is carried out by USB or WIRELESS connections or a password entered via the control panel (26) located inside the electronic control box (20).

By means of the counter (211) located on the electronic card and processor (21), the usage frequency of the stimulator device (A) by the patient and compatibility with treatment can be monitored by the doctor that is following up the patient.

The cooling compressor (20) and the gas perfluorocarbon tank (23) in the electronic control box (20) step in as soon as the stimulator device (A) creates an electromagnetic field and the electromagnetic coils (121) are cooled during the operation of the stimulator device (A) via the cooling cages (122) with +4° C. gas perfluorocarbon circulation. This cooling degree can be changed via the control panel (26) by the technical staff following the request of the doctor. The patient cannot control the cooling system and its temperature.

The electronic control box (20) is configured with a speaker (25) connected to an electronic card and processor (21), wherein said speaker (25) enables to guide the patient with audio commands in order for the patient who has visual impairment to be able to use the stimulator and iontophoresis device (A) alone, safely, correctly and efficiently. Thereby the audio guidance of the patient is provided with the speaker (25) following the detection by the magnetic field sensors (1212) connected to the electronic card and processor (21) that determine if the front cap (10a) is at a vertex distance to the eyeball or not, and if the electromagnetic coil assemblies (12) at the rear cap (10b) are at the correct contact distance symmetrically to the occipital cortex and inter-hemispheric regions. At the same time the starting of the treatment, ending of the treatment and remaining time are vocally notified to the patient via the speaker (25). Additionally by means of the electronic card and processor (21) the patient with low vision can be guided with commands in different languages.

The required parameters of the stimulator and iontophoresis device (A) for therapy, is efficient with various setting adjustments and is suitable for safe usage in order to prevent incorrect usage.

The magnetic field intensity at the electromagnetic coils (121), frequency, time and the electromagnetic coil (121) that is active, number of usages, and activation information of the stimulator and iontophoresis device (A) are collected via the counter (211) and such information can be seen on the screen (24) that is included in the electronic control box (20).

The electric cables (123) leading to the electromagnetic coil (121) and exiting out of the electronic control box (20) and the cooling pipes (124) between the cooling compressor (22) and the cooling cage (122) are housed as a group inside the connection pipe (30) that is positioned between the cap (10) and the electronic control box (20) such that they are insulated and isolated.

What is claimed is:

1. A stimulator and iontophoresis device used for treating retina and optical nerve diseases, comprising
a cap comprising a front cap and a rear cap, wherein the front cap comprises an electromagnetic coil assembly comprising electromagnetic coils, wherein the electromagnetic coils create four electromagnetic fields at a vertex distance to eyeballs, and each of the electromagnetic coils is covered with a cooling cage, and the rear cap comprises the electromagnetic coil assembly comprising the electromagnetic coils, wherein the electromagnetic coils create the four electromagnetic fields stimulating an occipital cortex region and one electromagnetic field stimulating an inter-hemispheric region, and each of the electromagnetic coils is covered with the cooling cage,
wherein the stimulator and iontophoresis device further comprises
a cooling compressor connected with cooling pipes passing through a connection pipe connected with cooling cages inside the front cap and the rear cap and a gas perfluorocarbon tank,
an electronic card and a processor having a counter, wherein the counter is provided on the processor, and the processor adjusts an intensity, a frequency and a time of each of the four electromagnetic fields created by the electromagnetic coils,
an electronic control box having a speaker connected to the electronic card and processor.

2. The stimulator and iontophoresis device according to claim 1, comprising a connection rail located at sides of the front cap and the rear cap, wherein the connection rail is configured to connect the front cap and rear cap to each other and to extend or shorten a distance between the front cap and the rear cap.

3. The stimulator and iontophoresis device according to claim 1, wherein the front cap and the rear cap are made of non-inflammable, antibacterial, antistatic medical plastic, carbon fibre or any other kind of medical material.

4. The stimulator and iontophoresis device according to claim 1, wherein sections besides the electromagnetic coil assembly at inner surfaces of the front cap and the rear cap are covered with a smart sponge rubber.

5. The stimulator and iontophoresis device according to claim 4, wherein the smart sponge rubber is made of a medical, inflammable, biocompatible, antiallergic and antibacterial material.

6. The stimulator and iontophoresis device according to claim 1, wherein each of the electromagnetic coils is polygonal and located inside the electromagnetic coil assembly within the front cap and the rear cap, wherein the electromagnetic coils create 2000 milligauss electromagnetic field for 30 minutes at 42 hertz frequency.

7. The stimulator and iontophoresis device according to claim 1, wherein the cooling cage is located on each of electromagnetic coils and the cooling cage prevents a risk of a thermal damage occurring at cellular levels on the eyeballs.

8. The stimulator and iontophoresis device according to claim 1, wherein the cooling cage is a double layered impermeable hollow box made of fibreglass, carbon fibre or any other medical material.

9. The stimulator and iontophoresis device according to claim 1, the cooling cage comprises a cooling tank allowing a gas circulation in the cooling cage, and further comprises a gas outlet hole and a gas inlet hole allowing entrance and discharging of a cooling gas.

10. The stimulator and iontophoresis device according to claim 1, wherein the cooling pipes are connected to the cooling compressor and the gas perfluorocarbon tank is located in the electronic control box, and the cooling pipes are further connected to the cooling cage in the front cap and the rear cap to enable a recirculation between a cooling tank and the cooling compressor comprised in the cooling cage.

11. The stimulator and iontophoresis device according to claim 1, comprising a control panel located in the electronic control box, wherein the control panel enables to adjust the intensity, the frequency and the time and an active-passive state of the electromagnetic coils via USB or wireless connections and at a same time, and the control panel enables to adjust a cooling degree of a cooling gas in the cooling cages.

12. The stimulator and iontophoresis device according to claim 1, wherein the counter is located in the electronic control box, and the counter enables a doctor following up a patient to monitor a treatment compatibility of the patient and a frequency of usage of the stimulator and iontophoresis device by the patient.

13. The stimulator and iontophoresis device according to claim 1, comprising a speaker connected to the electronic card and the processor, wherein the speaker enables to guide a patient with audio commands in different languages.

14. The stimulator and iontophoresis device according to claim 6, comprising a LED indicator located on the electromagnetic coils, wherein the LED indicator allows to track active and passive electromagnetic coils.

15. The stimulator and iontophoresis device according to claim 6, comprising magnetic sensors located on the electromagnetic coils, wherein the magnetic sensors reflect a magnetic field created when the electromagnetic coils are active onto a screen comprised in the electronic control box and at a same time to determine if the electromagnetic coil assembly is at a correct symmetrical contact distance to a patient.

16. The stimulator and iontophoresis device according to claim 1, comprising a screen located on the electronic control box, wherein the screen reflects, via the counter, collected information, wherein the collected information is a magnetic field intensity of the electromagnetic coils, the frequency, the time and activation of the electromagnetic coils, number of usages, and activation information.

17. The stimulator and iontophoresis device according to claim 1, wherein the connection pipe is positioned between the cap and the electronic control box, and the connection pipe houses as a group, the stimulator and iontophoresis device further comprises electric cables leading to the electromagnetic coils and exiting out of the electronic control box and the cooling pipes between the cooling compressor and the cooling cage to be insulated and isolated.

18. The stimulator and iontophoresis device according to claim 1, comprising a power supply located inside the electronic control box, wherein the power supply is a battery-accumulator and/or adapter operating directly with an electricity network, wherein the power supply is connected to the electromagnetic coils via electric cables.

19. The stimulator and iontophoresis device according to claim 6, wherein the cooling cage is located on the electromagnetic coils, and the cooling cage prevents a risk of a thermal damage occurring at cellular levels on the eyeballs.

20. The stimulator and iontophoresis device according to claim 7, wherein the cooling cage is a double layered impermeable hollow box made of fibreglass, carbon fibre or any other medical material.
